(19) United States
(12) Patent Application Publication
(10) Pub. No.: US 2024/0075174 A1
(43) Pub. Date: Mar. 7, 2024

(54) METHODS AND SYSTEMS FOR DECONTAMINATING A SURFACE USING GERMICIDAL UV LIGHT

(71) Applicant: U TECHNOLOGY CORPORATION, CALGARY (CA)

(72) Inventors: MARK LEFSRUD, Hudson (CA); REBECCA MARTINEZ, Dollard-Des-Ormeaux (CA); ANNE SOPHIE RUFYIKIRI, Ste-Anne-de-Bellevue (CA); SARAH MACPHERSON, Vaudreuil-Dorion (CA); BO-SEN WU, Saint-Laurent (CA)

(73) Assignee: U TECHNOLOGY CORPORATION, CALGARY, AB (CA)

(21) Appl. No.: 18/262,246

(22) PCT Filed: Jan. 27, 2022
(86) PCT No.: PCT/CA2022/050109

§ 371 Date: Jul. 20, 2023

Related U.S. Application Data
(60) Provisional application No. 63/142,526, filed on Jan. 28, 2021.

(51) Int. Cl. A61L 2/10

(52) U.S. Cl. CPC A61L 2/10; A61L 2202/11; A61L 2202/14

(57) ABSTRACT

A method of decontaminating a surface includes exposing the surface to germicidal UV light emitted by at least one germicidal UV light source, preferably a light emitting diode (LED), for a continuous exposure period of between 0.05 seconds and 2 seconds; discontinuing the exposure of the surface to the germicidal UV light for a continuous rest period of between 30 seconds and 120 seconds; and repeating steps a. and b. for a total of at least 3 cycles.

### CROSS-REFERENCES TO RELATED APPLICATIONS

This application claims the benefit of and/or priority to U.S. Provisional Patent Application No. 63/142,526 filed on Jan. 28, 2021, which is incorporated herein by reference in its entirety.

### FIELD

This document relates to decontamination. More specifically, this document relates to methods and systems for decontaminating surfaces using ultraviolet light.

### BACKGROUND

U.S. Pat. No. 9,179,703 (Shur et al.) describes directing ultraviolet radiation within an area. The target wavelength ranges and/or target intensity ranges of the sources of ultraviolet radiation can correspond to at least one of a plurality of selectable operating configurations, including a sterilization operating configuration and a preservation operating configuration.

U.S. Pat. No. 10,485,887 (Ramanand et al.) describes a pulsed UV disinfection system that includes a xenon UV lamp mounted in an articulated head assembly, and a chassis housing a high voltage power supply for driving the lamp and pulse configuration control unit for configuring the output of the power supply. The head assembly and the chassis are positioned on a mobile carriage. The pulse configuration control unit is programmed for driving the UV lamp at a rate of between 20 and 50 pulses per second, with each pulse emitting between 30 and 150 joules of UV radiant energy. The system also features remote video imaging of a target area, remote control of the carriage and head assembly as well as a remote emergency shutdown.

### SUMMARY

The following summary is intended to introduce the reader to various aspects of the detailed description, but not to define or delimit any invention.

Methods and systems for decontaminating a surface are disclosed.

According to some aspects, a method for decontaminating a surface includes exposing the surface to pulsed germicidal ultraviolet (UV) light emitted by at least one light source at a duty rate of at most 25% for at least 3 cycles. The light source is preferably a light emitting diode (LED).

In some examples, the duty rate is at most 10%. In some examples, the duty rate is at most 2%. In some examples, the duty rate is at most 1%.

In some examples, the surface is exposed for between 3 cycles and 150 cycles. In some examples, the surface is exposed for between 3 cycles and 100 cycles. In some examples, the surface is exposed for between 3 cycles and 50 cycles.

In some examples, the light is pulsed at a frequency of up to 20 Hz. In some examples, the light is pulsed at a frequency of between 0.5 Hz and 2 Hz. In some examples the light is pulsed at a frequency of about 1 cycle per minute.

In some examples, the germicidal UV light has a peak wavelength of between 220 and 320 nanometers. In some examples, the germicidal UV light has a peak wavelength of between 220 and 280 nanometers. In some examples, the germicidal UV light has a peak wavelength of between 270 and 280 nanometers. In some examples, the light source emits germicidal UV light at a peak wavelength of 222 nm. In some examples, the light source is an LED that emits germicidal UV light at a peak wavelength of 260 nm. In some examples, the germicidal UV light has a peak wavelength of 273 nm. In some examples, the germicidal UV light has a peak wavelength of 277 nm. In some examples, the germicidal UV light has a peak wavelength of 280 nm.

In some examples, the method includes measuring an air temperature in a vicinity of the surface and controlling the pulses of germicidal UV light based on the measured air temperature.

In some examples, the method includes measuring a relative humidity of air in a vicinity of the surface and controlling the pulses of germicidal UV light based on the measured relative humidity.

In some examples, the germicidal UV light is emitted at a fluence of between 0 and 100 mJ·cm⁻². In some examples, the fluence is between is 0 and 10 mJ·cm⁻².

In some examples, prior to exposing the surface to the pulsed germicidal UV light, the surface is contaminated with microorganisms, and exposing the surface to the pulsed germicidal UV light yields a log reduction of the microorganisms of at least 2. In some examples, the log reduction is at least 3. In some examples, the microorganisms include E. coli, B. subtilis, MS2 bacteriophage, and/or SARS-CoV-2.

According to some aspects, a system for decontaminating a surface includes a supply of power, at least one germicidal ultraviolet (UV) light source powered by the supply for exposing the surface to germicidal UV light, and a controller configured to control operation of the germicidal UV light source to cause the germicidal UV light source to emit the germicidal UV light in pulses at a duty rate of at most 25% for at least 3 cycles. The germicidal UV light source is preferably a light emitting diode (LED).

In some examples, the duty rate is at most 10%. In some examples, the duty rate is at most 2%. In some examples, the duty rate is at most 1%.

In some examples, the controller is configured to cause the germicidal UV light source to emit the germicidal UV light in pulses for between 3 cycles and 150 cycles. In some examples, the controller is configured to cause the light source to emit the germicidal UV light in pulses for between 3 cycles and 100 cycles. In some examples, the controller is configured to cause the light source to emit the germicidal UV light in pulses for between 3 cycles and 50 cycles.

In some examples, the controller is configured to cause the light source to emit the germicidal UV light at a frequency of up to 20 Hz. In some examples, the controller is configured to cause the germicidal UV light source to emit the germicidal UV light at a frequency of between 0.5 Hz and 2 Hz. In some examples, the controller is configured to cause the germicidal UV light source to emit the germicidal UV light at a frequency of about 1 cycle per minute.

In some examples, the germicidal UV light source emits germicidal UV light at a peak wavelength of between 220 and 280 nanometers. In some examples, the germicidal UV light source emits germicidal UV light at a peak wavelength of between 270 and 280 nanometers. In some examples, the UVC light source emits germicidal UV light at a peak wavelength of 222 nm. In some examples, the germicidal UV light source emits germicidal UV light at a peak wavelength of 260 nm. In some examples, the germicidal UV light source emits UVC light at a peak wavelength of 273 nm. In some examples, the germicidal UV light source emits germicidal UV light at a peak wavelength of 277 nm. In some examples, the germicidal UV light source emits germicidal UV light at a peak wavelength of 280 nm.

In some examples, the germicidal UV light source emits light at a fluence of between 0 and 100 mJ·cm⁻². In some examples, the germicidal UV light source emits light at a fluence of between is 0 and 10 mJ·cm⁻².

In some examples, the system further includes a temperature sensor configured to measure an air temperature in a vicinity of the surface. The controller can be configured to control the pulses of the germicidal UV light source based on the measured air temperature.

In some examples, the system further includes a humidity sensor configured to measure a relative humidity of air in a vicinity of the surface. The controller can be configured to control the germicidal UV light source based on the measured relative humidity.

According to some aspects, a method of decontaminating a surface includes: a. exposing the surface to germicidal ultraviolet (UV) light emitted by at least one germicidal UV light source for a continuous exposure period, b. discontinuing the exposure of the surface to the germicidal UV light for a continuous rest period that is longer than the exposure period, and c. repeating steps a. and b. The germicidal UV light source is preferably a light emitting diode (LED).

In some examples, the continuous rest period is at least double the continuous exposure period. In some examples, the continuous rest period is at least ten times the continuous exposure period. In some examples, the continuous rest period is at least sixty times the continuous exposure period.

In some examples, the continuous exposure period summed with the continuous rest period yields a cycle time, the continuous exposure period divided by the cycle time yields a duty rate, and the duty rate is at most 25%. In some examples, the duty rate is at most 10%. In some examples, the duty rate is at most 2%. In some examples, the duty rate is at most 1%.

In some examples, step c. includes repeating steps a. and b. for a total of at least 3 cycles. In some examples, step c. includes repeating steps a. and b. for a total of between 3 cycles and 150 cycles. In some examples, step c. includes repeating steps a. and b. for a total of between 3 cycles and 100 cycles. In some examples, step c. includes repeating steps a. and b. for a total of between 3 cycles and 50 cycles.

In some examples, the germicidal UV light has a peak wavelength of between 220 and 280 nanometers. In some examples, the germicidal UV light has a peak wavelength of between 270 and 280 nanometers. In some examples, the germicidal UV light source emits germicidal UV light at a peak wavelength of 222 nm. In some examples, the germicidal UV light source emits germicidal UV light at a peak wavelength of 260 nm. In some examples, the germicidal UV light has a peak wavelength of 273 nm. In some examples, the germicidal UV light has a peak wavelength of 277 nm. In some examples, the germicidal UV light has a peak wavelength of 280 nm.

In some examples, the method further includes measuring an air temperature in a vicinity of the surface and controlling the pulses of germicidal UV light based on the measured air temperature.

In some examples, the method further includes measuring a relative humidity of air in a vicinity of the surface and controlling the pulses of germicidal UV light based on the measured relative humidity.

In some examples, the germicidal UV light is emitted at a fluence of between 0 and 100 mJ·cm⁻². In some examples, the fluence is between is 0 and 10 mJ·cm⁻².

In some examples, the method yields a log reduction in microorganisms on the surface of at least 2. In some examples, the log reduction is at least 3. In some examples, the microorganisms include E. coli, B. subtilis, MS2 bacteriophage, and/or SARS-CoV-2.

According to some aspects, a method of decontaminating a surface includes exposing the surface to germicidal UV light having a peak wavelength of between 220 and 280 nm emitted by at least one light emitting diode (LED) for a continuous exposure period of between 0.05 seconds and 2 seconds, discontinuing the exposure of the surface to the germicidal UV light for a continuous rest period of between 30 seconds and 120 seconds, and repeating steps a. and b. for a total of at least 3 cycles.

### BRIEF DESCRIPTION OF THE DRAWINGS

The drawings included herewith are for illustrating various examples of articles, methods, and apparatuses of the present specification and are not intended to limit the scope of what is described in any way. Throughout the present description reference will be made to the following drawings, in which:

FIG. 1 is a schematic diagram of an example system for decontaminating a surface.

FIG. 2 is a flow chart illustrating an example method for decontaminating a surface.

FIG. 3 is a graph showing the relative spectra of two UV lamps and a number of UV light sources as measured with a spectroradiometer (PS-300, Apogee, corrected but uncalibrated below 380 nm).

FIG. 4 shows the germicidal efficacy of UV radiation (log 10 reduction in CFU or PFU/dosage) on a) E. coli, b) B. subtilis endospores, c) MS2 bacteriophage and d) a SARS-CoV-2 isolate irradiated with a KrCl excimer lamp (222-nm), UV gas discharge lamp (255-nm), and UV-LEDs with peak wavelengths at 260 nm, 273 nm, 277 nm, and 280 nm.

FIG. 5 shows Log reduction of E. coli with pulsed UV light under the same fluence at different duty rates. Duty rates were 0.33% (0.2 sec/60 sec, 150 cycles), 0.5% (0.3 sec/60 sec, 100 cycles), 0.83% (0.5 sec/60 sec, 60 cycles), 1.67% (1 sec/60 sec, 30 cycles), 16.67% (10 sec/60sec, 3 cycles), and 20% (10 sec/50 sec, 3 cycles. Fluence for each wavelength was as follows: 18.90 mJ·cm⁻² for 222-nm KrCl lamp, 2.66 mJ·cm⁻² for 260-nm UV-LED, 16.78 mJ·cm⁻² for 273-nm UV-LED, 63.40 mJ·cm⁻² for 277-nm UV-LED, and 60.84 mJ·cm⁻² for 280-nm UV-LED.

FIG. 6 shows Log reduction of B. subtilis with pulsed UV light under the same fluence at different duty rates. Duty rates were 0.33% (0.2 sec/60 sec, 150 cycles), 0.5% (0.3 sec/60 sec, 100 cycles), 0.83% (0.5 sec/60 sec, 60 cycles), 1.67% (1 sec/60 sec, 30 cycles), 16.67% (10 sec/60 sec, 3 cycles) and 20% (10 sec/50 sec, 3 cycles). Fluence for each wavelength was as follows: 18.90 mJ·cm⁻² for 222 nm, 2.66 mJ·cm⁻² for 260 nm, 16.78 mJ·cm⁻² for 273 nm, 63.40 mJ·cm⁻² for 277 nm, and 60.84 mJ·cm⁻² for 280 nm.

FIG. 7 shows Log reduction of SARS-COV-2 PFU with pulsed UV light with the same fluence with different duty rates. Duty rates were 1.7% (1 sec/60 sec, 30 cycles) and 20% (10 sec/50 sec, 3 cycles). Fluence for each wavelength was as follows: 18.90 mJ·cm⁻² for 222-nm KrCl lamp and 63.40 mJ·cm⁻² for 277-nm UV-LED.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are methods and systems that utilize germicidal ultraviolet (UV) light, such as ultraviolet-C (UVC) light, from one or more light sources, preferably one or more light emitting diodes (LEDs), to decontaminate surfaces.

As used herein, the term “decontamination” can refer to both the reduction of microorganisms (e.g., viruses such as coronavirus, bacteria such as e-coli, and fungi) on a surface (i.e. “disinfection”), as well as the killing or all or substantially all microorganisms on the surface (i.e. “sterilization”). The methods and systems described herein can be used to decontaminate surfaces of objects having different sizes, uses and materials, such as but not limited to electronics (e.g., mobile telephones, tablets, laptop computers), food and agricultural products (e.g., fruit and vegetables), clothing and wearable objects (e.g., personal protective equipment including masks, gowns, face shields), medical devices (e.g., surgical tools, thermometers, stethoscopes, blood pressure monitors, haemostats, scissors), hospital equipment (e.g., ventilator components, carts, stretchers, household and personal objects (e.g., keys), office supplies, (e.g., pens), grooming tools (e.g., nail and hair clippers, tweezers), cosmetics (e.g., makeup brushes), furniture and appliances (e.g. tabletops and countertops), fixtures (e.g. doorknobs and toilet seats) and others.

In the methods and systems described herein, the surface is exposed to germicidal UV light, such as UVC light, for a continuous exposure period, followed by a continuous rest period in which the surface is not exposed to the germicidal UV light (e.g. the germicidal UV light source may be turned off). This cycle of exposure and rest is repeated, for example for at least 3 cycles, or for between 3 and 150 cycles, or for between 3 and 100 cycles, or for between 3 and 50 cycles. Notably, the exposure period can be relatively short, while the rest period can be relatively long. For example, the exposure period can be about 10 seconds or less, or about 2 seconds or less, or about 1 second or less, or about 0.1 seconds, or about 0.2 seconds, or about 0.3 seconds, or about 0.5 seconds, or about 0.001 seconds, while the rest period can be about 50 seconds or more, or about 58 seconds or more, or about 59 seconds or more, or about 59.9 seconds, or about 59.8 seconds, or about 59.7 seconds, or about 59.5 seconds, or about 59.009 seconds. For further example, the rest period can be at least double the exposure period, or at least 10 times the exposure period, or at least sixty times the exposure period. For further example, the germicidal UV light can be emitted a duty rate of at most about 25%, or of at most about 10%, or of at most about 2%, or of at most about 1%, or of about 0.17%, or of about 0.33%, or of about 0.50%, or of about 0.83%, or of about 1.67% (where the duty rate is expressed as a percentage and is calculated as the exposure period divided by the cycle time, where the cycle time is calculated as the exposure period summed with the rest period). For further example, the frequency of the cycle may be up to about 20 Hz, or up to about 10 Hz, or between about 0.5 Hz and about 2 Hz, or between 0 and 1 Hz, or about 1 cycle per minute (i.e. about 0.0167 Hz). In some preferred examples, the exposure period is about 1 second or less, the rest period is about 59 seconds or more, the duty rate is about 2% or less, the frequency is about 1 cycle per second, and the cycle of exposure and rest is repeated about 150 or fewer times.

Surprisingly, it has been determined that even with relatively short exposure periods and relatively long rest periods, decontamination of surfaces can be achieved. Even further surprisingly, it has been determined that in some instances, shortening the exposure period can achieve more effective decontamination. For example, in some instances, germicidal UV light emitted at a duty rate of 16.67 achieved a log reduction in microorganisms of about 2, while germicidal UV light emitted at a duty rate of 1.67 achieved a log reduction in microorganisms of about 4.

The cycle of exposure and rest described above may also be described as “pulsed” emission of germicidal UV light.

As used herein, the term “germicidal UV light” refers to ultraviolet light having a peak wavelength of between about 220 nm and about 320 nm, inclusive. In some examples, the germicidal UV light can be UVC light, and can have a peak wavelength of between about 220 nm and about 280 nm. For example, the UVC light can have a peak wavelength of between about 260 nm and about 280 nm, or between about 270 nm and about 280 nm, or about 222 nm, or about 260 nm, or about 273 nm, or about 277 nm, or about 280 nm. In some examples, the germicidal UV light can be UVB light, and can have a wavelength of between about 280 nm and about 320 nm. For example, the UVC light can have a peak wavelength of about 310 nm or about 320 nm.

In some examples, the germicidal UV light can be emitted at fluences of between 0 and 100 mJ·cm⁻², or of between 0 and 10 mJ·cm⁻².

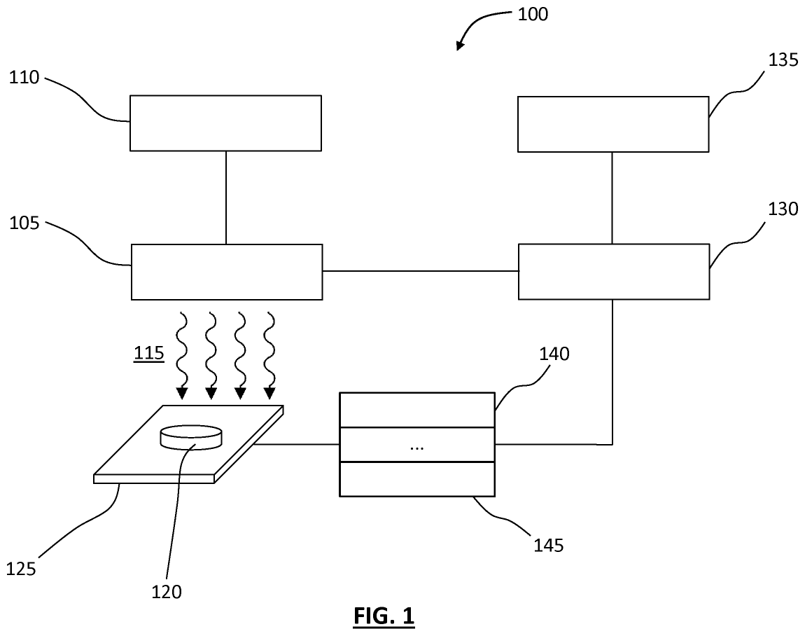

Referring now to the drawings, FIG. 1 shows an example system 100 that utilizes UVC light to decontaminate surfaces. In the example shown, system 100 includes a UVC LED array 105 powered by a suitable supply of power 110, such as a 120V or 240V AC source or by a battery. As described above, the UVC LED array 105 emits light 115 having a peak wavelength falling within the ultraviolet-C (UVC) range of the spectrum onto a surface of an object 120, which can be placed, for example, on a supporting surface 125 within a decontamination device or apparatus so as to decontaminate the surface of the object 120.

The UVC LED array 105 may include a plurality of light emitting diodes (LEDs), which may be arranged, for example, in a one- or two-dimensional grid or pattern. The LEDs may be, for example, fabricated using aluminum nitride (AIN), aluminum gallium nitride (AlGaN), aluminum gallium indium nitride (AlGaInN), or diamond substrate technologies among others. In alternative examples, a single UVC LED may replace the UVC LED array.

As described above, the LEDs of the UVC LED array 105 may emit light 115 having a peak wavelength in the UVC range (a peak wavelength of between about 220 nm and 280 nm). More specifically, in some examples, light 115 may have a peak wavelength between about 220 nm and about 280, or between about 270 nm and about 280 nm, or about 222 nm, or about 260 nm, about 273 nm, or about 277 nm, or about 280 nm.

The UVC LED array 105 may include UVC LEDs of a single peak wavelength in the UVC range. Alternatively, the UVC LED array 105 may include UVC LEDs of multiple peak wavelengths in the UVC range.

System 100 may also include a controller 130 that is configured generally to control operation of the UVC LED array 105 to cause the UVC LED array 105 to emit light 115 in pulses. A user interface 135 coupled to the controller 130 may be used to input one or more different control parameters into controller 130 to be used in the control of UVC LED array 105. For example, such input parameters may include any or all of pulse frequency (i.e. the number of cycles of exposure and rest per second), duty rate, total number of cycles, selection of peak wavelength(s) (where UV LED array 105 contains LEDs of multiple different peak wavelengths), irradiance, or output level of the LEDs.

Where UVC LED array 105 contains LEDs of multiple different wavelengths, controller 130 may also allow for a selection of one or more target wavelengths and, if applicable, a relative proportion of each emitted wavelength. The UVC LED array 105 may then be controlled to emit light 115 of the selected wavelength(s).

In some examples, system 100 may also include one or more sensors to detect conditions on or in the vicinity of object 120 and provide detection data to controller 130 to be used in the control of UVC LED array 105. For example, system 100 may in some examples include a temperature sensor 140 that detects an air temperature in the vicinity of object 120 and provides a signal encoding this data to controller 130. In some examples, system 100 may include a humidity sensor 145 that detects a relative humidity of the air in the vicinity of object 120 and provides a signal encoding this data to controller 130.

In some embodiments, UVC LED array 105 and/or system 100 as a whole may be mounted on or as part of a decontamination apparatus, such as is described in United States Patent Application Publication No. 2021/0338863 (Hammad et al.), the entirety of which is incorporated herein by reference. In such cases, object 120 may be placed in an enclosure within a suitable decontamination apparatus wherein object 120 is exposed to pulses of light 115 emitted from UVC LED array 105. Without limitation, for example, UVC LED array 105 can be mounted within such an enclosure or in some other location on a decontamination apparatus such that object 120 is exposed to the light 115 emitted by the UVC LED array 105. Additionally, in some examples, controller 130 and user interface 135 may be integrated within a decontamination apparatus but can also be implemented as standalone devices that are electronically coupled to UVC LED array 105 and/or supply 110.

Based on, for example, input parameters received from user interface 135, controller 130 may control the flow of power from supply 110 to UVC LED array 105 to cause the UVC LED array 105 to emit light 115 in pulses for an exposure period followed by a rest period, at a duty rate, at a frequency, and/or for a specified duration of time or number of cycles.

As described above, the exposure period can be about 30 seconds or less, or about 2 seconds or less, or about 1 second or less, or about 0.1 seconds, or about 0.2 seconds, or about 0.3 seconds, or about 0.5 seconds, while the rest period can be about 50 seconds or more, or about 58 seconds or more, or about 59 seconds or more, or about 59.9 seconds, or about 59.8 seconds, or about 59.7 seconds, or about 59.5 seconds.

As described above, the duty rate may be, for example, at most about 25%, or at most about 10%, or at most about 2%, or at most about 1%, or about 0.17%, or about 0.33%, or about 0.50%, or about 0.83%, or about 1.67%.

The frequency may be, for example, up to about 20 Hz, or up to about 10 Hz, or between about 0.5 Hz and about 2 Hz, or between 0 and 1 Hz, or about 1 cycle per minute (i.e. about 0.0167 Hz).

The number of cycles may be, for example, between 3 and 150 cycles, or between 3 and 100 cycles, or between 3 and 50 cycles, or about 30 cycles, or about 50 cycles.

The controller 130 may additionally instruct UVC LED array 105 to emit any set number of pulses or to emit pulses for any set duration of time.

As described above, it has surprisingly been determined that even with relatively short exposure periods and relatively long rest periods, decontamination of surfaces can be achieved. In addition, the use of pulsed germicidal UV emission may be relatively cost effective as it both uses less energy overall and generates less source heat, leading to longer LED life (e.g. exceeding 100,000 hours operation). That is, in some examples, the lifespan of system 100 may be relatively long, yet system 100 may emit large UVC radiation levels, as by operating system 100 at a relatively low duty rate, the junction temperature of the UVC array may be kept relatively low. Furthermore, because energy output is lower overall in comparison to continuous emission, the exposure risk to humans may be minimized. For example, there may be lower risk of accidental UV exposure to lab personnel and technicians working in the vicinity of the germicidal UV LEDs. For further example, photobiology eye safety may be enhanced, as the actinic UV exposure limit (e.g. 30 Jm⁻², 8 h daily, EU directive 2006/25/EC) may be avoided.

The selection of emission parameters for light 115, such as exposure period and rest period, duty rate, frequency, number of pulses, and/or emission wavelength, may be made manually through receipt of input commands on user interface 135. Alternatively, the selection may be made automatically by controller 130 and, optionally, adjusted based on detection signals received from temperature sensor 140 or humidity sensor 145.

In some examples, the number of exposure periods or the length of the exposure period may be selected by controller 130 or inputted through user interface 135 so that UVC LED array 105 delivers a target fluence of light 115 to object 120. The fluence level at which effective disinfection or sterilization of object 120 occurs may be relatively low. In some cases, for example, UVC LED array 105 may deliver a fluence of between 0 and 100 mJ·cm⁻² or, more particularly, of between 0 and 20 mJ·cm⁻² or even as low as between 0 and 5 mJ·cm⁻².

In some examples, system 100 may include one or further components. One such component is a lens or a waveguide (not shown) used to focus or direct the emission of light 115 onto the surface of the object 120. Another such component is a cooling apparatus (e.g. a fan or a heat sink). Another such component is a source of visible light (not shown) that may be controlled by controller 130 to operate simultaneously when the UVC LED array 105 is on. Because UVC light is generally invisible to the human eye, simultaneous emission of visible light can be used as an indicator or warning that system 100 is operational and emitting potentially dangerous UV radiation.

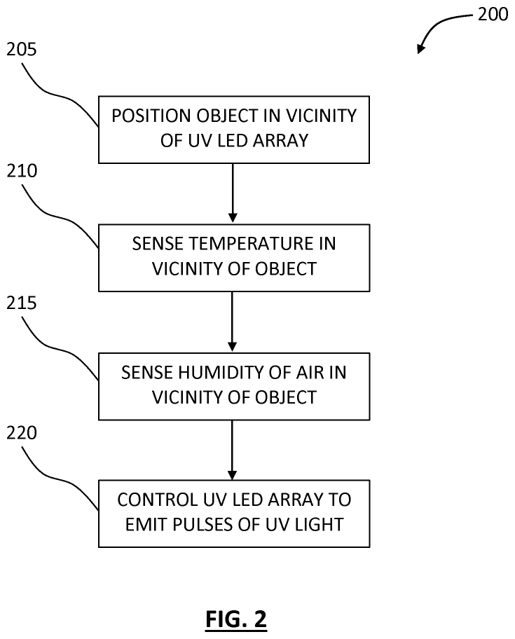

FIG. 2 illustrates a method 200 that may be used to control operation of a germicidal UV light source. The method 200 may be performed, for example, by or in conjunction with a system 100. Unless the contrary is expressly stated or implied by context, parts and sequences of method 200 as described may be altered, varied, performed in a different order, or omitted altogether.

In the example shown, at step 205, an object having a surface to be decontaminated may be positioned proximate one or more UVC LEDs that may be included in, for example, a UVC LED array. For example, an object may be placed in a decontamination apparatus such as is described in United States Patent Application Publication No. 2021/0338863. In other cases, a UVC LED array may be supported apart from a decontamination apparatus, such as on a free-standing frame inside a room.

At step 210, in the example shown, an air temperature may be sensed in the vicinity of the object to be irradiated. Further, at step 215, in some cases, a relative humidity of the air in the vicinity of the object maybe measured. These (and other) readings may then be provided to a controller or other system component for use in controlling the operation of a UV LED array. In some cases, steps 210 and 215 may be omitted from method 200.

At step 220, a UV LED array may emit pulses of UVC light onto the surface of the object to be decontaminated, to thereby expose microorganisms on the surface to UVC light. The parameters of the UVC light, such as peak wavelength, exposure period and rest period, frequency, duty rate, cycle length, and fluence, may be as described above.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

### EXAMPLES

### Materials and Methods

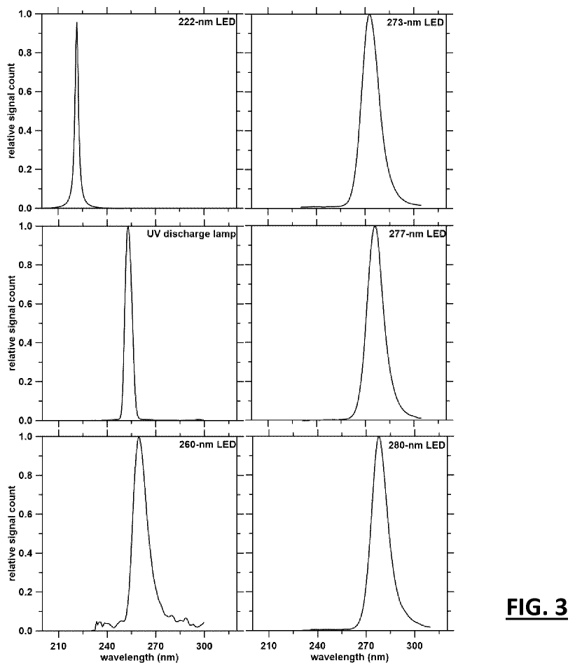

Experimental apparatus. A gas-based UV lamp incorporated into a laminar flow hood (254-nm peak wavelength, Forma Scientific, Thermo Forma 1845, Waltham, US), a 222-nm KrCl excimer lamp (Excimer UVC-222, Guandong Excimer Optoelectronic Co., Jiangmen City, China) and four UV-LED light strips emitting different peak wavelengths across the UV-C range were tested. UV-LEDs emitted 260-nm (U Technology Corporation, Calgary, Canada), 273-nm (U Technology Corporation, Calgary, Canada), 277-nm (EHC Global Inc., Oshawa, Canada, and U Technology Corporation, Calgary, Canada), and 280-nm (EHC Global Inc., Oshawa, Canada) wavelengths. FIG. 3 shows the spectra of the UV LEDs as determined with a spectroradiometer (PS-300, Apogee, Logan, UT). To measure intensity, UV-LED strips were connected to a power supply (DP832, Rigol Tech, Beaverton, OR, US) and secured face down with clamps in a laminar flow hood. Heat sinks (Advanced Thermal Solutions Inc., Norwood, MA, US) were incorporated into the experimental setup for the 260-nm and 273-nm LEDs to allow for heat dissipation, but this was not possible for the 277-nm and 280-nm LED configurations. LEDs were turned on and allowed to stabilize (5-10 min). UV light source (UV-LEDs or UV lamps) intensity outputs and coverage areas were measured and mapped at room temperature (23° C.) using an UV sensor (ILT770-UV, International Lighting Technology). LED intensities (irradiance outputs) were measured prior to each germicidal test to confirm uniformity of testing parameters between replications and treatments. Spectral error of the UV radiation meter for each UV-LED configuration was calculated as described previously (Ross & Sulev, 2000, Wu & Lefsrud, 2018). Briefly, a 278-nm LED light spectrum was used as a reference spectrum to obtain the corrected irradiance outputs of the UV radiation meter. For the gas-based lamp incorporated into the laminar flow hood, irradiance output was measured at different predetermined distances from the lamp. The apparent and corrected irradiance levels, fluences, and corresponding wattage outputs for each UV light source is summarized below in Table 1.

Bacterial Strains, Viral Inocula, And Culture Preparation. The disinfection and sterilization efficiency of germicidal UV radiation was investigated on BCL1 Gram-negative Escherichia coli, Bacillus subtilis endospores, SARS-CoV-2, and a positive-stranded RNA bacteriophage, MS2, according to modified protocols described previously by Ortega, et al. (2007), Kim, et al. (2017), and Welch, et al. (2018). Escherichia coli (ATCC 15597; C-3000 derived from K-12), Bacillus subtilis (ATCC 23857), and Escherichia coli bacteriophage MS2 (ATCC 15597-61; host E. coli C-3000) were obtained (Cedarlane, Burlington, ON), and stock cultures were kept frozen at −77° C. and maintained on Luria-Bertani (LB; 1° A peptone, 0.5% yeast extract, and 1% NaCl)-agar (1.5%) plates. A SARS-CoV-2 isolate (CP13.32 P3, MUHC, March 2020) was propagated and titered in Vero E6 cells. Viral stocks were stored at −80° C.

Single colony-forming units (CFU) of E. coli were picked from an agar plate to start overnight cultures in LB (25 ml/125-ml Erlenmeyer flask), shaking with 200 RPM at 37° C. After 24 h, the optical density of the overnight E. coli culture was measured using a spectrophotometer (Ultrospec 2100, Biochrom, Cambridge, UK). Cells were washed and resuspended in phosphate buffered saline (PBS; 137 mM NaCl, 10 mM Phosphate, 2.7 mM KCl, pH 7.4) to obtain 10⁸ colony forming units (CFU)/mL.

For B. subtilis, endospores were prepared as described by Tavares (2013), with some modifications. Difco sporulation media was inoculated (25 ml/125 mLErlenmeyer flask) with 2-3 CFU and grown for 96 h at 37° C. Cells were pelleted at 10,00 RPM and resuspended in PBS containing 50 μg lysozyme/ml and incubated 1 h@37° C., followed by 10 min at 80° C. Cells were pelleted for 5 min at 10,000 RPM, washed three times with water and resuspended in PBS. Endospores were confirmed with malachite green/safranin staining and light microscopy.

MS2 was reconstituted in LB as per manufacturer's instructions and aliquots were kept frozen at −75° C. Aliquots were thawed on ice and diluted 10-fold in LB prior to irradiation. SARS-CoV-2 viral stock was thawed on a cold block and diluted to 100,000 PFU/ml prior to irradiation.

UV irradiation and fluence. Prior to irradiation, 500 μL E. coli cell suspension, 500 μL B. subtilis endospore suspension, or 180 μL freshly diluted MS2 inoculum (stock diluted 10-fold in LB) were placed as a single droplet in an uncovered 100 mm Petri dish within a laminar flow hood, where ultraviolet UV sources were set up to irradiate the pathogens at predetermined fluence for each UV source, as described further below.

UV irradiation was determined by measuring light intensity (uW cm⁻²) with a spectroradiomenter, and different exposure times were used to increased fluence (dosage). LEDs were secured with clamps and oriented to face down toward the center of the pathogen-containing droplets placed on 100-mm Petri dishes (see above). Fluence (mJ cm⁻²) was calculated by multiplying light intensity×total exposure time. As such, fluence for all six UV sources were set as follows: 222-nm (356-2138 mJ cm⁻²) 254-nm (2202-13212 mJ cm⁻²), 260-nm (319-1844 mJ cm⁻²), 273-nm (440-2657 mJ cm⁻²), 277-nm (1270-7618 mJ cm⁻²), and 280-nm (1245-7471 mJ cm⁻²). Given containment level 3 (CL3) facility time constraints, determination of germicidal efficacy against the SARS-CoV-2 isolate was limited to the 222-nm and 277-nm UV sources. Pulsed lighting treatments were performed with a DC power supply (DP832, Rigol Tech., Beaverton, OR, USA) together with an Arduino (Arduino, Somerville, MA, USA) for the 222 nm, 260 nm, and 273 nm UV light sources. For the 277 nm and 280 nm LEDs, a controller provided by the manufacturer (EHC Global) was employed for radiation output and pulse control. The pathogen-containing droplets were treated with pulsed UV light at different duty rates with same aggregate fluence over time. Tables 1 and 2 below lists the parameters used for pulsed UV light, along with continuous radiation at the same fluence as control (baseline).

TABLE 1
Irradiance levels of the UV module and corresponding
fluences in pulsed UV radiation experiment.

| | Highest irradiance | | | |
|---|---|---|---|---|
| UV Peak | level (μW · cm⁻²) | | Fluence | Wattage |
| Wavelength | Apparent | Corrected | (mJ · cm⁻²) | (W) |
| 222-nm | 300 | 603 | 18.90 | 12 |
| 255-nm | 1750 | 1750 | 1050 | 30 |
| 260-nm | 63 | 88 | 2.66 | 1.92 |
| 273-nm | 522 | 559 | 16.78 | 9.6 |
| 277-nm | 2100 | 2116 | 63.40 | 0.04 |
| 280-nm | 2100 | 2122 | 60.84 | 0.04 |

TABLE 2
Examined duty rates (percent of one cycle that light is
on) along with corresponding exposure times and cycles.

| | Exposure Period per | |
|---|---|---|
| Duty rate (%) | minute (sec) | Total cycles |
| 0.17 | 0.1 | 150 |
| 0.33 | 0.2 | 150 |
| 0.50 | 0.3 | 100 |
| 0.83 | 0.5 | 60 |
| 1.67 | 1.0 | 30 |
| 16.67 | 10 | 3 |
| 100 | — | — |
| (continuous lighting) | | |

Un-irradiated controls included the same volumes of E. coli and B. subtilis endospore suspensions, or diluted MS2 inocula placed in an uncovered Petri dish for the same duration without any laminar hood illumination.

The same method was performed with a diluted stock of the SARS-CoV-2 isolate (CP13.32 P3), and 220-uL droplets were placed in a Petris dish in a biological safety cabinet at McGill University's BCL3 facility. The same volume (un-irrradiated) was placed on a Petri dish and served as a control for the same duration. Cold blocks were used to manipulate the virus during serial dilutions.

After irradiation, the Petri dish containing the irradiated E. coli, B. subtilis endospore suspension, or diluted MS2 inoculum was rinsed several times before being transferred to an Eppendorff. If volume was lost to drying, sterile PBS (or LB for MS2) was added to the irradiated E. coli and B. subtilis cell suspensions, or the MS2 inocula to reach the pre-irradiation volume. Triplicate serial dilutions were performed in PBS for E. coli and B. subtilis, or LB for MS2. For E. coli and B. subtilis, 100 μL of select dilutions were spread on 100 mm Petri dishes containing LB-agar and incubated overnight at 37° C. For MS2, plaque assays were performed using a modified double layer agar technique (Kauffman & Polz (2018). Briefly, 100 μL of an E. coli overnight culture (host) and 100 μL of each selected serial MS2 dilution were dropped on an LB-agar Petri dish, followed by the addition of 2-3 mL molten LB-0.3% agar. The mixture was quickly mixed by swirling several times and evenly spread over the surface of the dish. Top agar solidified for 20 min at RT before incubating overnight at 37° C. CFU and plaque forming units (PFU) were counted the next day with OpenCFU 3.8 image processing software (Gueissman, 2013). Counts were manually verified prior to determining logarithmic reductions in CFU or PFU.

Plaque enumeration for SARS-CoV-2 was performed according to Mendoza et al (2020), by infecting a Vero E6 cell monolayer, carried out with 12-well plates and crystal violet staining. Testing was temporally replicated three times for each UV light source and fluence.

Reduction calculation. Reductions (CFU) for each treatment were determined based on the following equation:

$$Log Reduction = {log}_{10} ( \frac{A}{B} ) \qquad ( 1 )$$

where A represents the CFU of the sample before treatment and B represents the CFU of the sample after treatment.

### Results

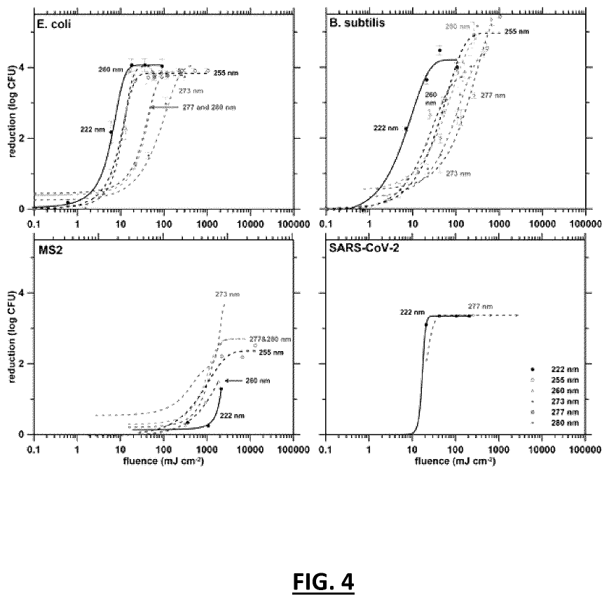

Continuous Germicidal UV Light. FIG. 4 shows the measured reduction of pathogens treated with germicidal UV light emitted continuously from the UV LEDs and UV lamps at different fluences (mJ·cm⁻²).

The highest log-reduction in E. coli CFU with minimal fluence was 4.07±0.16 (99.991% reduction) with the 222-nm KrCl UV lamp (18 mJ cm−2). This was followed by a 3.89±0.14 log reduction (99.987% reduction) with the 260-nm UV-LED (53 mJ cm⁻²), 3.94±0.06 (99.989% reduction) with 273-nm UV-LED (221 mJ cm⁻²), 4.25±0.25 (99.994% reduction) with the 277-nm UV-LED (300 mJ cm⁻²), and a 3.88±0.25 log-reduction (99.987% reduction) with the 280-nm UV-LED (125 mJ cm⁻²). This compared to a 3.9±0.13 log reduction (99.987% reduction) obtained using the control UV discharge lamp (255-nm at 262 mJ cm⁻²).

The highest log-reduction in B. subtilis endospores with minimal fluence was 4.47±0.13 (99.996% reduction) with the 222-nm KrCl UV lamp (42 mJ cm⁻²). This was followed by a 3.87±0.25 log reduction (99.986% reduction) with the 260-nm UV-LED (160 mJ cm⁻²), 4.36±0.08 (99.996% reduction) with 273-nm UV-LED (221 mJ cm⁻²), 5.35±0.17 (99.9995% reduction) with the 277-nm UV-LED (738 mJ cm⁻²), and a 5.16±0.38 log-reduction (99.9993% reduction) with the 280-nm UV-LED (311 mJ cm⁻²). This compared to a 5.44±0.06 log reduction (99.9996% reduction) obtained using the control UV discharge lamp (255-nm at 1050 mJ cm⁻²).

The highest log reduction in MS2 PFU with minimum UV fluence was 2.7±0.09 (99.8% reduction) with the 277-nm UV-LED at 3809 mJ cm⁻². Log reductions were comparable for the 273-nm (2.54±0.04 log reduction with 2657 mJ cm⁻²) and the 280-nm (2.45±0.15 log reduction with 7471 mJ cm⁻²) UV-LEDs. The 222-nm KrCl lamp and the 260-nm UV-LED obtained similar log-reductions, 1.29±0.33 with 2138 mJ cm⁻² and 1.65±0.28 with 1844 mJ cm⁻², respectively. This compared to a 2.21±0.56 log reduction (99.38% reduction) obtained using the control UV discharge lamp (255-nm at 2202 mJ cm⁻²).

Surface disinfection of a SARS-COV-2 isolate with only two UV modules was investigated. Using this method, a log reduction of 3.24 (99.94% reduction) was obtained with the 222 KrCl UV lamp emitting 6.67 mJ cm−2. It is believed that log reductions >3.24 may be obtained with the 222-nm KrCl UV lamp at higher fluence (13-180 mJ cm⁻²), as no PFU were detected in the plaque assay with these UV conditions. A comparable log reduction of 3.96 (99.989% reduction) was obtained with the 277-nm UV-LED with nearly 10 times the UV fluence (60 mJ cm⁻²). It is believed that a log reduction of >3.96 may be obtained with the 277-nm UV LED at higher fluence (120-600 mJ cm⁻²), as no PFU were detected in the plaque assay with these UV conditions.

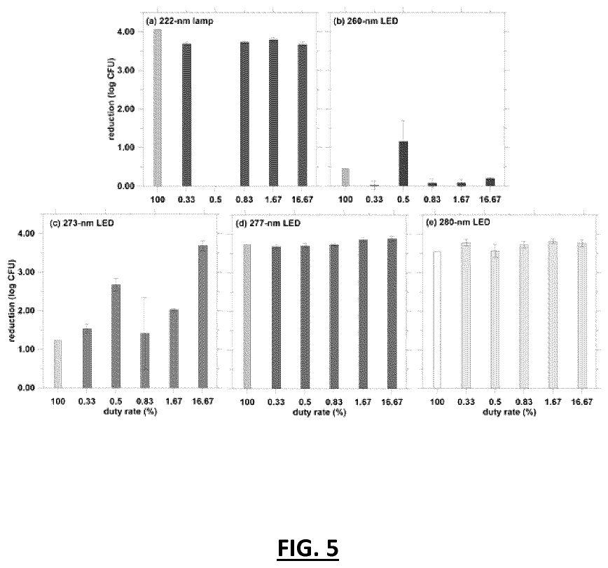

Pulsed UV Light. The impact of pulsed UV light at different duty rates (Table 2) was investigated on E. coli, B. subtilis endospores, and a SARS-CoV-2 isolate. UV modules were the same as tested for continuous UV irradiation: 222-nm (KrCl UV lamp), 260-nm (UV-LED), 273-nm UV-LED, 277-nm (UV-LED), and 280-nm (UV-LED). Logarithmic reductions in CFU (E. coli and B. subitilis) or PFU (SARS-CoV-2) are plotted against UV pulse treatment for each UV light; data are summarized in FIGS. 5 to 7.

For E. coli (FIG. 5), the highest log reduction in CFU for pulsed irradiation was 3.8 (99.98% reduction) for the 277-nm UV-LED at a duty rate of 20% and 1.67%. At duty rates of 0.33%, 0.5% and 0.83%, log reductions of 3.6, 3.6 and 3.7 were reached for 277 nm, respectively. For 280-nm (UV-LED), duty rates of 1.67, 20, 0.83, and 0.5% resulted in log reductions of 3.7, 3.6, 3.6, and 3.6, respectively. The lowest log reduction for 277 nm was 2.5 at a duty rate of 0.33%. UV light from the 222 nm KrCl lamp nearly reached a 4-log-reduction at a duty rate of 1.67%. Duty rates of 0.83, 0.33 and 16.67 resulted in log reduction of 3.7. For 273 nm, a log reduction of 3.7 was achieved at a duty rate of 16.67%; a log reduction of 2.3 was achieved at a duty rate of 0.5%; a log reduction of 2 was achieved at a duty rate of 1.67; a log reduction of 1.5 was achieved at a duty rate of 0.33%; and a log reduction of 1.4 was achieved at a duty rate of 0.83%. The wavelength with the lowest log reduction was 260-nm (UV-LED), with a 0.1 log reduction at a duty rate of 0.33%. Log reductions of 0.2, 0.2, and 0.3 were obtained with duty rates of 0.83, 1.67 and 16.67%, respectively. The highest log reduction for 260 nm was 1.5 at a duty rate of 0.5%.

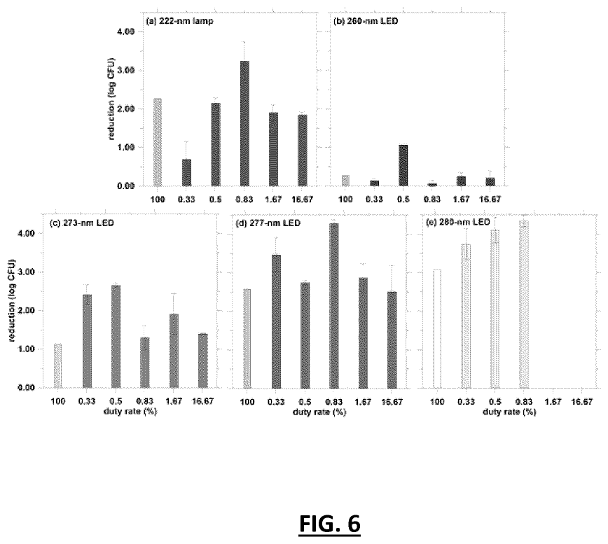

For B. subtilis (FIG. 6), the 280-nm UV-LED resulted in the highest log reduction of 4.2 (99.994% reduction) at a duty rate of 0.83%. Duty rates of 0.5% and 0.33% had log reductions of 4.0 and 3.7, respectively. For 277-nm (UV-LED), a similar 4.2 log reduction at a duty rate of 0.83% was observed. Duty rates of 0.33, 0.5, 1.67, and 16.67% resulted in log reductions of 3.5, 2.7, 2.80 and 2.5, respectively. For 273 nm, the duty rate at 0.5% resulted in the highest log reduction 2.7 (99.80% reduction). Duty rates 0.33%, 1.67%, 16.67%, and 0.83% had a log reduction of 2.4, 1.9, 1.4, and 1.3, respectively. The 222-nm KrCl lamp showed a log reduction of 3.2 (99.937% reduction) at a duty rate of 0.83%, a log reduction of 1.8 at a 1.67% duty rate, a log reduction of 1.7 at a 16.67% duty rate, a log reduction of 2.1 at 0.5%, and a log reduction of 0.7 at a 0.33% duty rate.

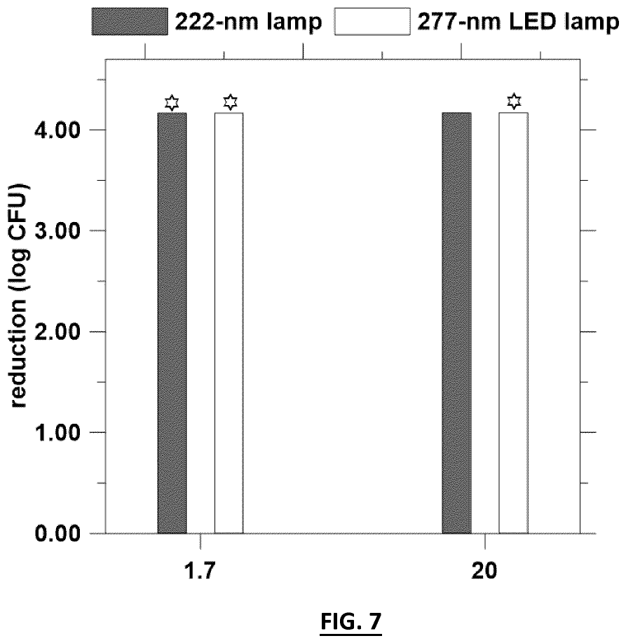

For SARS-COV-2 (FIG. 7), two pulsing UV modules emitting the same fluence at different duty rates of 1.7% (1 sec/60 sec, 30 cycles) and 20% (10 sec/50 sec, 3 cycles) were investigated (FIG. 3.3). A log reduction of 4.17 (99.993% reduction) was obtained with the 222 KrCl UV lamp at a 1.7% duty rate (18 mJ cm−2). It is believed that log reductions>4.17 can be obtained using the 222-nm KrCl UV lamp with a 20% duty rate, as no PFU were detected in the plaque assay with these UV conditions. Based on the data obtained, it is believed that a log reduction of >4.17 can be obtained with the 277-nm UV-LED, at both 1.7% and 20% duty rates emitting 65 mJ cm⁻², as no PFU were detected in the plaque assay with these UV conditions. Thus, in FIG. 7, the star indicates that log reductions of greater than 4.17 (>99.993% reduction) can be obtained using the 222-nm KrCl UV lamp with a 1.7% duty rate, and with the 277-nm UV-LED, bot at 1.7% and 20% duty rates, as no PFU were detected in the plaque assay with these UF conditions.

### REFERENCES

Ross, J. & Sulev, M. Sources of errors in measurements of PAR. Agricultural and Forest Meteorology 100, 103-125 (2000).

Wu, B.-S. & Lefsrud, M. G. Photobiology eye safety for horticultural LED lighting: Transmittance performance of eyewear protection using high-irradiant monochromatic LEDs. Journal of occupational and environmental hygiene 15, 133-142 (2018).

Geissmann, Q. OpenCFU, a new free and open-source software to count cell colonies and other circular objects. PloS one 8 (2013).

Ortega, M., Franken, L., Hatesohl, P. & Marsden, J. Efficacy of ecoquest radiant catalytic ionization cell and breeze at ozone generator at reducing microbial populations on stainless steel surfaces. Journal of Rapid Methods Automation in Microbiology 15, 359-368 (2007).

Kim, D.-K., Kim, S.-J. & Kang, D.-H. Bactericidal effect of 266 to 279 nm wavelength UVC-LEDs for inactivation of Gram positive and Gram negative foodborne pathogenic bacteria and yeasts. Food Research International 97, 280-287 (2017).

Welch, D. et al. Far-UVC light: A new tool to control the spread of airborne-mediated microbial diseases. Scientific Reports 8, 1-7 (2018).

Kauffman, M. & Polz, F. Streamlining standard bacteriophage methods for higher throughput. MethodsX 5, 159-172 (2018).

Mendoza, E. J., Manguiat, K., Wood, H. and Drebot, M., 2020. Two detailed plaque assay protocols for the quantification of infectious SARS-CoV-2. Current protocols in microbiology, 57(1), p.cpmc105.

Tavares, M. B., Souza, R. D., Luiz, W. B., Cavalcante, R. C., Casaroli, C., Martins, E. G., Ferreira, R. C. and Ferreira, L. C., 2013. Bacillus subtilis endospores at high purity and recovery yields: optimization of growth conditions and purification method. Current microbiology, 66(3), pp.279-285.

1. A method of decontaminating a surface, comprising:
exposing the surface to pulsed germicidal UV light emitted by at least one light source at a duty rate of at most 25% for at least 3 cycles.

2. The method of claim 1, wherein the germicidal UV light source is a light emitting diode.

3. The method of claim 1 or claim 2, wherein the duty rate is at most 10%.

4. (canceled)

5. (canceled)

6. The method of claim 1, wherein the surface is exposed for between 3 cycles and 150 cycles.

7. (canceled)

8. (canceled)

9. The method of claim 1, wherein the germicidal UV light is pulsed at a frequency of up to 20 Hz.

10. (canceled)

11. (canceled)

12. The method of claim 1, wherein the germicidal UV light has a peak wavelength of between 220 and 280 nanometers.

13. (canceled)

14. The method of claim 1, wherein the germicidal UV light has a peak wavelength of 222 nm, of 260 nm, or 273 nm, or 277 nm, or 280 nm.

15. (canceled)

16. (canceled)

17. (canceled)

18. (canceled)

19. The method of claim 1, further comprising measuring an air temperature in a vicinity of the surface and controlling the pulses of germicidal UV light based on the measured air temperature, and/or measuring a relative humidity of air in the vicinity of the surface and controlling the pulses of germicidal UV light based on the measured relative humidity.

20. (canceled)

21. The method of claim 1, wherein the germicidal UV light is emitted at a fluence of between 0 and 100 mJ·cm⁻².

22. (canceled)

23. The method of claim 1, wherein prior to exposing the surface to the pulsed germicidal UV light, the surface is contaminated with microorganisms, and exposing the surface to the pulsed germicidal UV light yields a log reduction of the microorganisms of at least 2.

24. (canceled)

25. The method of claim 1, wherein the microorganisms comprise E. coli and/or SARS-COV-2.

26. (canceled)

27. A system for decontaminating a surface comprising:
a. a supply of power;
b. at least one germicidal UV light source powered by the supply for exposing the surface to UVC light; and
c. a controller configured to control operation of the germicidal UV light source to cause the germicidal UV light source to emit the germicidal UV light in pulses at a duty rate of at most 25% for at least 3 cycles.

28. The system of claim 27, wherein the germicidal UV light source is a light emitting diode (LED).

29. The system of claim 27, wherein the duty rate is at most 10%.

30. (canceled)

31. (canceled)

32. The system of claim 27, wherein the controller is configured to cause the light source to emit the germicidal UV light in pulses for between 3 cycles and 150 cycles.

33. (canceled)

34. (canceled)

35. The system of claim 27, wherein the controller is configured to cause the germicidal UV light source to emit the germicidal UV light at a frequency of up to 20 Hz.

36. (canceled)

37. (canceled)

38. The system of claim 27, wherein the light source emits germicidal UV light at a peak wavelength of between 220 and 280 nanometers.

39. (canceled)

40. (canceled)

41. (canceled)

42. (canceled)

43. (canceled)

44. (canceled)

45. The system of claim 27, wherein the germicidal UV light source emits germicidal UV light at a fluence of between 0 and 100 mJ·cm⁻².

46. (canceled)

47. The system of claim 27, further comprising a temperature sensor configured to measure an air temperature in a vicinity of the surface, wherein the controller is configured to control the pulses of the germicidal UV light source based on the measured air temperature, and/or a humidity sensor configured to measure a relative humidity of air in the vicinity of the surface, wherein the controller is configured to control the germicidal UV light source based on the measured relative humidity.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. A method of decontaminating a surface, comprising:
exposing the surface to ultraviolet-C (UVC) light having a peak wavelength of between 200 and 280 nm emitted by at least one light emitting diode (LED) for a continuous exposure period of between 0.05 seconds and 2 seconds;
discontinuing the exposure of the surface to the UVC light for a continuous rest period of between 30 seconds and 120 seconds; and
repeating steps a. and b. for a total of at least 3 cycles.
